(19) United States
(12) Patent Application Publication
(10) Pub. No.: US 2011/0071469 A1
(43) Pub. Date: Mar. 24, 2011

(54) Single Use Retractable Infusion or Transfusion Needle

(76) Inventors: Craig Douglas Wilson, New South Wales (AU); Ian Patrick Berrell, New South Wales (AU)

(21) Appl. No.: 12/672,275

(22) PCT Filed: May 1, 2008
(86) PCT No.: PCT/AU08/00593

§ 371 Date: Feb. 5, 2010

(30) Foreign Application Priority Data
Aug. 13, 2007 (AU) 2007904326

(51) Int. Cl. A61M 5/50

(52) U.S. Cl. 604/110

(57) ABSTRACT

A needle assembly (10) has a hollow tubular housing (12) and a needle sub assembly (14) including a needle (18) slideably mounted therein. Two diametrically opposed legs (30) extend radially outwards and backwards from the needle shaft (16) and engage the rear end (42) of the housing (12) surrounding an aperture (74) through which the rear of the shaft (16) extends. The legs hold the subassembly (14) in an extended state against the action of a spring (52). Two diametrically opposed arms (70) overlie the legs (30) when the needle sub assembly (14) is extended and which may be squeezed together to compress the legs (30) inwards and allow the legs (30) to pass through the aperture (74) under the action of the spring (52) allowing the needle subassembly (14) to retract and draw the point (26) of the needle (18) into the housing (12).

### FIELD OF INVENTION

This invention relates to retractable needles and more particularly to single use retractable infusion or transfusion needles. However, the invention is not limited to infusion or transfusion needles

### BACKGROUND

Needle stick injuries are a serious health risk for health professionals. Single use retractable infusion or transfusion needles are known. These tend to require manual extraction of the needle from the patient.

U.S. Pat. No. 5,573,512 to van den Haak discloses a retractable infusion or transfusion needle assembly having a needle sub assembly sliceable within housing. The needle sub assembly is manually withdrawn from a patient into the housing or sheath. The user must grasp the housing and manually withdraw the cannula from the patient and into the housing whilst holding the housing against the patient's skin and preventing its movement. Whilst the needle sub assembly is locked in position once withdrawn and cannot be re-extended, if the user slips the entire assembly may be withdrawn, with the cannula point exposed and subsequent risk of needle stick injury. Further, it is possible for the user to easily twist or otherwise disturb the orientation of the cannula in the patient's vein during this extraction process, with subsequent risk of injury to the patient.

Auto-retracting needles have been proposed. However they suffer from constructions that allow auto-retraction to easily occur accidentally, particularly as a needle is being prepared for insertion or during insertion. If auto retraction accidentally occurs the needle and any other equipment attached to the needle, such as a whole blood collection bag, must be thrown away and a new assembly used.

### SUMMARY OF THE INVENTION

To overcome at least some of the disadvantages of the prior art the present invention provides a single use retractable infusion or transfusion needle that enables a user to simultaneously withdraw a needle from a patient and into a housing without needing to manually withdraw the cannula for the patient but in which accidental auto-retraction is prevented or substantially reduced.

In one broad form the invention provides a needle assembly including:
an elongate housing having a first aperture in a front end portion thereof and a second aperture in a rear end portion thereof;
an elongate hollow needle sub assembly slideably mounted within the elongate housing and having a needle point at a first end portion thereof and a second end portion adapted to be connected to a supply tube;
the needle sub assembly movable relative to the housing between an extended position, wherein the first end portion extends out of the housing through the first aperture and the needle point is located outside of the housing and a retracted position wherein the first end portion and the needle point is located and shielded within the housing;
biasing means biasing the needle sub assembly to the retracted position;
holding means having an operative position that, when the needle sub assembly is in the extended position, holds the needle sub assembly in the extended position against the action of the biasing means and prevents movement the needle sub assembly toward the retracted position;
release means movable to a release position for releasing the holding means from the operative position to allow movement from the extended to the retracted position;
locking means for locking the needle sub assembly when in the retracted position and preventing movement of the needle sub assembly toward the extended position to expose the needle point, and
means for preventing accidental activation of one or both of the release means or the holding means.

In one form the means for preventing accidental activation of the release means comprises blocking means that prevents movement of the release means toward the release position.

In another form the means for preventing accidental activation of the release means may comprise the release means itself and the release means may comprise a plurality of release members that must all be moved to a release position to release the holding means; when less than all of the plurality of release members are in the release position the holding means is not released.

In another form the means for preventing accidental activation of the release means may include both the blocking means and multiple release members.

In another broad form the invention provides a needle assembly including:
an elongate housing having a first aperture in a front end portion thereof and a second aperture in a rear end portion thereof;
an elongate hollow needle sub assembly slideably mounted within the elongate housing and having a needle point at a first end portion thereof and a second end portion adapted to be connected to a supply tube;
the needle sub assembly movable relative to the housing between an extended position, wherein the first end portion extends out of the housing through the first aperture and the needle point is located outside of the housing and a retracted position wherein the first end portion and the needle point is located and shielded within the housing;
biasing means biasing the needle sub assembly to the retracted position;
holding means having an operative position that, when the needle sub assembly is in the extended position, holds the needle sub assembly in the extended position against the action of the biasing means and prevents movement the needle sub assembly toward the retracted position;
release means movable to a release position for releasing the holding means from the operative position to allow movement from the extended to the retracted position;
locking means for locking the needle sub assembly when in the retracted position and preventing movement of the needle sub assembly toward the extended position to expose the needle point, and
at least one movable blocking member that, in a blocking position, prevents movement of the release means to the release position.

In yet another broad form the invention provides a needle assembly including:
an elongate housing having a first aperture in a front end portion thereof and a second aperture in a rear end portion thereof;
an elongate hollow needle sub assembly slideably mounted within the elongate housing and having a needle point at a first end portion thereof and a second end portion adapted to be connected to a supply tube;
the needle sub assembly movable relative to the housing between an extended position, wherein the first end portion extends out of the housing through the first aperture and the needle point is located outside of the housing and a retracted position wherein the first end portion and the needle point is located and shielded within the housing;
biasing means biasing the needle sub assembly to the retracted position;
holding means having an operative position that, when the needle sub assembly is in the extended position, holds the needle sub assembly in the extended position against the action of the biasing means and prevents movement the needle sub assembly toward the retracted position;
locking means for locking the needle sub assembly when in the retracted position and preventing movement of the needle sub assembly toward the extended position to expose the needle point, and
at least two release members movable to a release position for releasing the holding means from the operative position to allow movement from the extended to the retracted position,
said the holding means not released from the operative position unless all of the at least two release members are at the release position.

The locking means preferably includes at least one first leg that extends from one of the needle sub assembly and the housing and engages engagement means on the other of the needle sub assembly and the housing. Preferably the free end of the at least one first leg engages the engagement means.

Preferably the at least one first leg is resilient and is bent, flexed or otherwise distorted to allow movement to the retracted position. However, the at least one first leg may be relatively rigid and movable from the operative position. This may be by a pivoting or sliding action, for example. The at least one first leg may be biased to the locked or unlocked position, either by an external biasing member or by being resilient and bent away from the relaxed state.

The at least one first leg and engagement means are preferably configured so that the needle sub assembly can move from the extended position to the retracted position but not the other way. Preferably at least one first leg is angled to the longitudinal direction.

Preferably the at least one first leg is located on the housing and extends rearwards and radially inwards toward the needle sub assembly to engage engagement means on the needle sub assembly. Alternatively, the at least one first leg may be located on the needle sub assembly and extends forwards and radially outwards from the needle sub assembly to engage engagement means on the housing.

Preferably the at least one first leg comprises at least two opposed first legs that extend toward the needle sub assembly. Where two first legs are provided they need not be opposed and may, for example, be located at 90 degrees to each other. Multiple first legs may be provided.

The engagement means is preferably at least one flange or protrusion. However the engagement means may be at least one recess.

Preferably the engagement means is part of the needle sub assembly. More preferably the engagement means comprises a flange.

The shaft is preferably tubular and preferably the flange is a radially extending annular flange. However, the shaft and/or the flange may have a non circular end cross section.

The holding means preferably includes at least one second leg that extends from one of the needle sub assembly and the housing and engages second engagement means on the other of the needle sub assembly and the housing. Preferably the free end of the at least one second leg engages the second engagement means.

Preferably the at least one second leg is resilient may be bent, flexed or otherwise distorted to allow movement to the retracted position. However, the at least one second leg may be relatively rigid and movable from the operative position. This may be by a pivoting or sliding action, for example. The at least one second leg may be biased to the locked or unlocked position, either by an external biasing member or by being resilient and bent away from the relaxed state.

Preferably at least one second leg is angled to the longitudinal direction.

Preferably the at least one second leg is located on the needle sub assembly and extends rearwards and radially outwards toward the housing to engage engagement means on the housing. Alternatively, the at least one second leg is located on the housing and extends forwards and radially inwards toward the needle sub assembly to engage engagement means on the needle sub assembly.

Preferably the at least one second leg comprises two opposed legs that extend from the needle sub assembly. Where two second legs are provided they need not be opposed and may, for example, be located at 90 degrees to each other. Multiple first legs may be provided.

The second engagement means is preferably at least one flange or protrusion. However the engagement means may be at least one recess.

Preferably the engagement means is part of the housing. More preferably the engagement means comprises the rear end of the housing and most preferably the portion of the housing surrounding the second aperture.

Preferably the second aperture is sized to allow the second legs to pass through when deflected to the release position.

Preferably the second aperture is sized so the flange cannot pass therethrough. Preferably the second aperture is rectangular.

The release means preferably include at least one portion for moving the holding means to the release portion. Preferably the at least one portion is resilient and is flexed to move the holding means. However, the at least one portion may be relatively rigid and movable from the operative position. This may be by a pivoting or sliding action, for example. The at least one portion may be biased to the release or non release position, either by an external biasing member or by being resilient and bent away from the relaxed state.

The at least one portion is preferably part of the housing.

Preferably the assembly includes blocking means for blocking movement of the release means to the release position.

Preferably the assembly includes at least one volume into which the release means moves to release the holding means.

Preferably the blocking means blocks movement of the release means into the at least one volume. Preferably the blocking means extends into at least part of the volume.

In the preferred form, the housing comprises a hollow tubular body and the needle sub assembly comprises a hollow shaft having a needle mounted at one end thereof. The holding means comprises two diametrically opposed legs that extend radially outwards and backwards from the shaft that engage the rear end of the housing surrounding an aperture through which the rear of the shaft extends. The housing has two diametrically opposed arms that overlie the legs when the needle sub assembly is extended and which may be squeezed together to compress the legs inwards and allow the legs to pass through the aperture. A spring located between the front portion of the housing and a flange on the shaft urges the shaft backwards when the holding legs are released. The housing has two opposed locking legs that extend rearwards and inwards to lock the needle sub assembly in the retracted position. The legs engage the front surface of the flange on the shaft against which the spring bears. Preferably the legs on the housing are located adjacent the arms but positioned at 90 degrees circumferentially to the arms.

The invention shall be better understood from the following description of non-limiting examples.

### BRIEF DESCRIPTION OF THE DRAWINGS

FIG. 1 is a perspective view from above of a first example of the invention in an extended position.

FIG. 2 is an exploded perspective view from above of the first example.

FIG. 3 is a side view of the first example in an extended position.

FIG. 4 is a cross sectional side view from the side of the first example in an extended position.

FIG. 5 is a detail cross sectional side view from the side of part of the first example in an extended position.

FIG. 6 is a top view of the first example in an extended position.

FIG. 7 is a cross sectional view from above of the first example in an extended position.

FIG. 8 is a detail cross sectional view from above of part of the first example in an extended position.

FIG. 9 is an end view of the first example.

FIG. 10 is an axial cross sectional view of the first example taken along line AA in FIG. 6, with a safety locking clip shown, which is not shown in FIG. 6.

FIG. 11 is a perspective view from above of a first example of the invention in a retracted position.

FIG. 12 is a cross sectional side view from the side of the first example in a retracted position.

FIG. 13 is a detail cross sectional side view from the side of part of the first example in a retracted position.

FIG. 14 is a cross sectional view from above of the first example in a retracted position.

FIG. 15 is an exploded perspective view from above of a whole blood needle according to a second example of the invention.

FIG. 16 is a perspective view from above of a variation of a needle sub assembly that may be used in the housing of the first example.

FIG. 17 is a detail cross sectional side view from the side of part of a needle assembly including the needle sub assembly of FIG. 16.

FIG. 18 is a perspective view from above of a needle assembly according to a further example of the invention.

FIG. 19 is a perspective view from above of the needle assembly of FIG. 18 activated to allow retraction.

FIG. 20 is an axial cross sectional view from above of the example of FIG. 18 in a retracted position.

FIG. 21 is an exploded perspective view from above of a dialysis needle according to a further example of the invention.

FIG. 22 is a perspective cross sectional view of the example of FIG. 21 in an extended position.

FIG. 23 is a perspective cross sectional view of the example of FIG. 21 in an extended position but activated to allow retraction.

FIG. 24 is a perspective cross sectional view of the example of FIG. 21 in a retracted position

### DETAILED DESCRIPTION OF EXAMPLES OF THE INVENTION

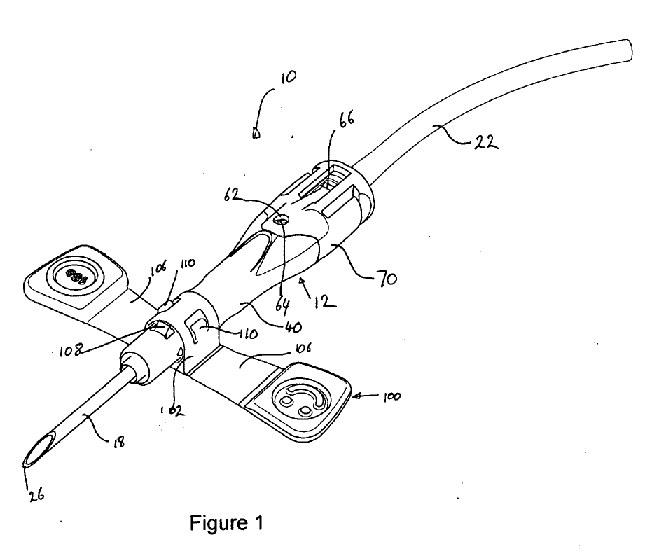

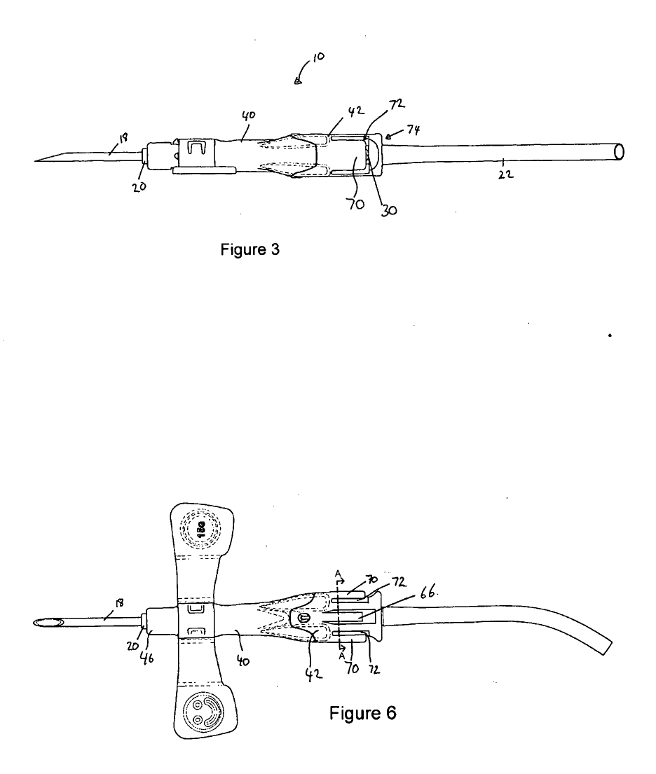

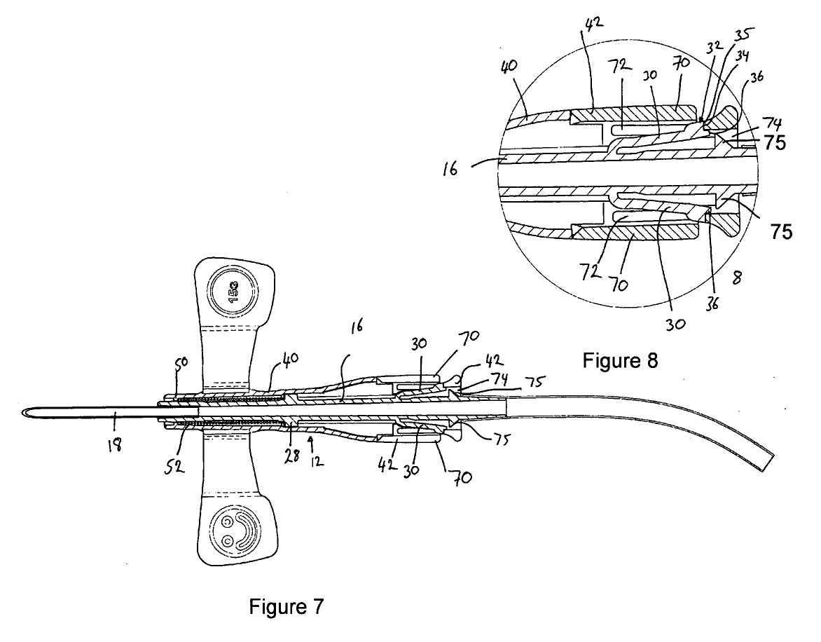

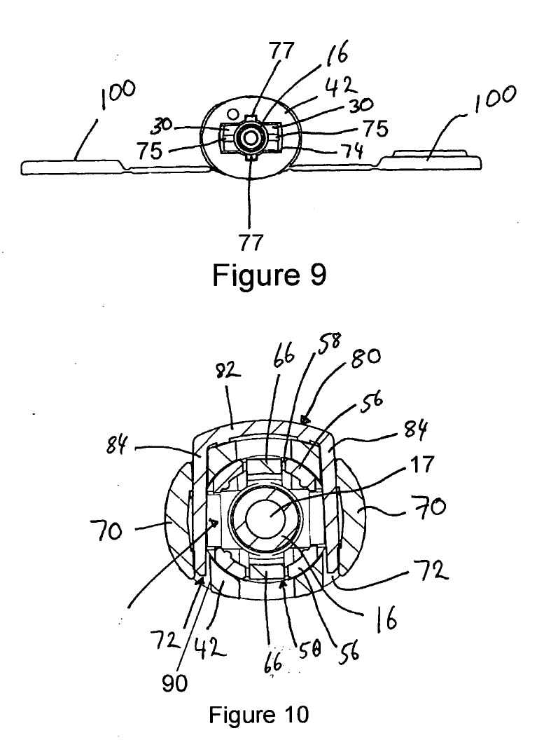

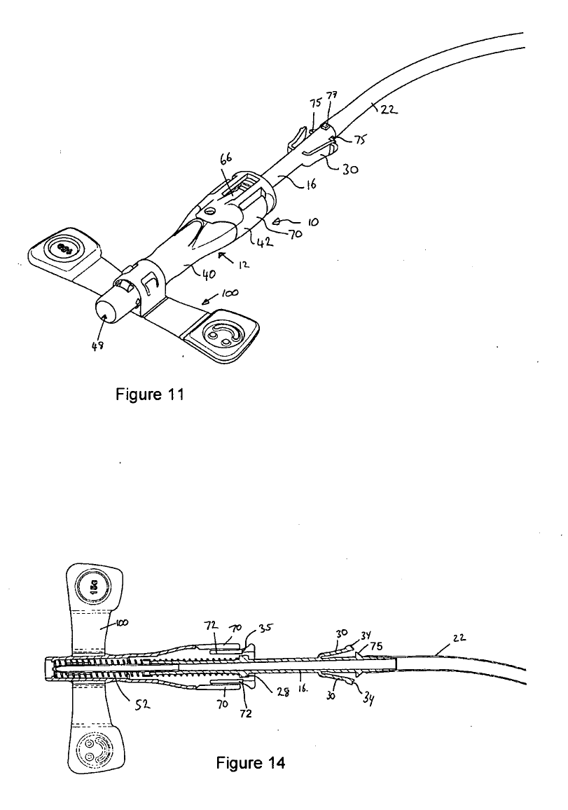

Referring to FIGS. 1 to 14 there is shown a first example of a retractable infusion or transfusion needle assembly 10 according to a first example of the invention. The retractable needle assembly 10 has an elongate housing 12 in which a needle sub assembly 14 is movable between an extended position, shown in FIG. 1 and a retracted position, shown in FIG. 11.

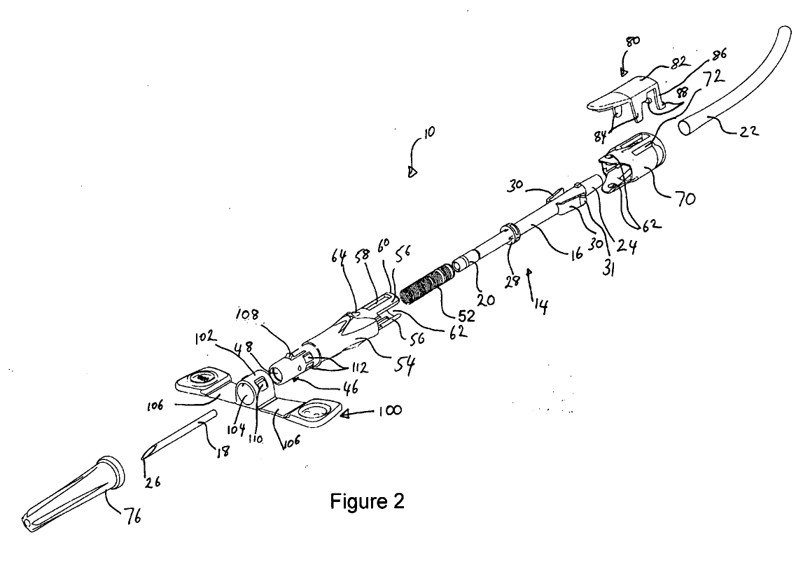

The needle sub assembly (see FIG. 2) comprises a hollow shaft 16 having a cannula 18 mounted at a front end 20. A plastics tube 22 may be mounted at the other end 24. Fluid may thus be supplied via the plastics tube 22 and bore 17 of hollow shaft 16 to the cannula 18 or vice versa. In the extended position the point 26 of the cannula 18 extends out of housing 12 and may be inserted into a patient's vein. In the retracted position the cannula, and in particular its point 26, is located within the housing 12 and is not exposed to the user. In the example substantially the entire cannula 18 extends out of the housing 12 in the extended position, but this is not essential.

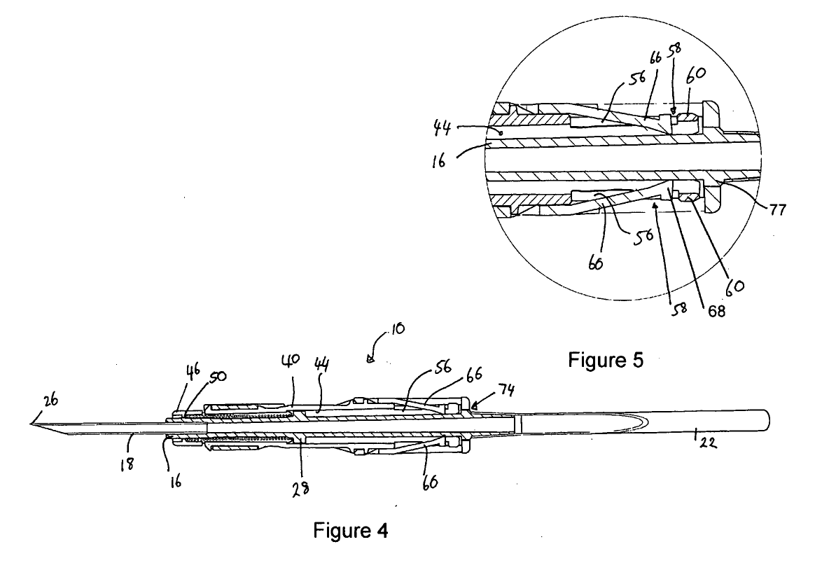

Preferably the cannula 18 is mounted within the end 20 of the shaft 16, as seen in FIG. 4. In this example the cannula is glued in the end of the shaft. This may also be achieved by injection moulding the shaft 16 around the cannula 18 during manufacture. The exact nature of the connection of the cannula 18 to the shaft 16 is not critical.

The shaft 16 has an annular flange 28 located between its ends. The length of the shaft 16 and the location of the flange 28 depend on the length of the housing 12.

Located toward the upper end 24 of the shaft 16 are two resilient legs 30. These legs 30 extend backwards and away from the axis of the shaft 16 and (as seen in FIG. 8) at their free ends 32 have a surface 34 extending generally perpendicular to the axis of the shaft 16. A small protrusion 36 extends backwards beyond the surface 34. The legs 30 are flexible and may be bent toward the shaft 16 on application of an inwards directed force, springing back when the force is released.

The housing 12 comprises a main body 40 and an end body 42. The main body 12 is elongate and has a generally cylindrical bore 44 within which the cannula 18 and front portion of shaft 16 are located. The bore 44 is preferably sized to be a snug fit with the flange 28 on the shaft 16 but to still allow free movement of the shaft within the bore 44. The front end 46 of the main body 40 has a circular aperture 48. The aperture 48 is sized to allow the cannula 18 and the end 20 of the shaft to extend through the aperture, as seen in FIG. 4. If desired the aperture 48 may be sized so that only the cannula 18 may extend out of the aperture 48.

A spring 52 is located around the shaft 16 and extends between the flange 28 on the shaft 16 and a flange 50 adjacent the aperture 48. The spring 52 has an outer diameter smaller than that of the flange 28 and so leaves an outer portion of the flange extending radially more than the spring 52. The flange 50 need not be adjacent the aperture 48 and, if desired, may be located away from the aperture 48. The spring 52 is more compressed in the extended position than in the retracted position and biases the shaft 16 and cannula 18 away from the front end 46.

The bore 44 may be tapered toward the aperture 48 so that movement of the needle subassembly out of the housing 12 is limited by the flange 28 engaging the surface of the bore 44. Movement of the needle sub assembly may also be limited by maximum compression of the spring or by other means.

The rear end 54 of the main body has two parallel arms 56 located on either side of the axis, best seen in FIG. 2. Each arm 56 has a longitudinally extending slot 58. Each slot 58 is closed by cross piece 60 of arm 56. The two arms 58 also define two vertical slots 62 between themselves. These slots 62 are sized so that the legs 30 on the shaft 16 may slide into and out of these slots.

The end body 42 is sized to slide over and lock to the rear end of the main body. The end body is preferably formed of a plastic and has apertures 62 that are a snap fit on protrusions 64 on the main body 40. In this position the end body 42 surrounds the arms 56 and the slots 62. Other forms of securing the end body 42 to the main body 40 may be used. If desired the end body 42 may be permanently affixed to the main body 40 after assembly be various means, such as gluing, welding or the like.

The end body 42 has a first pair of diametrically opposed resilient legs 66 that extend rearwards and inwards toward the longitudinal axis. When assembled the legs 66 extend into the slots 58. The free ends 68 of these legs 66 extend inwards further than the outer diameter of the central annular flange 28 on shaft 16 and preferably engage with and are biased against the surface of the shaft 16. In the extended position the flange 28 is located between the legs 66 and the front end 46.

Located at 90 degrees to the first pair of legs 66 are a second pair of diametrically opposed resilient legs 70 that extend rearwards. The legs extend generally parallel to the shaft. Each leg 70 defines a slot 72 between itself and the body of the end body 42. The slots 72 allow the legs 70 to be deflected inwards with a squeezing action. When assembled the legs 70 are located adjacent the slots 62 of the main body. When the needle sub assembly is in the extended position the legs 30 on the shaft 16 lie in the slots 62 and, preferably, the free end 31 of each leg 30 engages a corresponding leg 70. This is not essential and in this position there may be free space between the respective legs 30 and 70. The positioning of the legs 30 within slots 62 when in the extended position prevents the needle sub assembly turning relative to the housing 14. Thus if the tube 22 places a twisting force on the shaft 16, this will transferred to the patient via legs 30 and housing 14 rather than via the cannula 18. Thus twisting of the cannula 18 in the patient is resisted and/or prevented.

If desired, the bore 44 in the main body may have a non circular cross section and the flange 28 may have a corresponding non circular cross section, thereby preventing relative rotation about the longitudinal axis.

The end of the end body 42 has a rectangular opening 74. The width of this opening 74 is sized so that the legs 30 on the shaft cannot pass through the opening in their undeflected state. The legs 30 can only pass through the opening if deflected toward the shaft 16. The height of opening 74 is less than the outside diameter of the flange 28 and the so flange cannot pass through the opening 74.

The example has a wing assembly 100 mounted on the front end 46 of the main body 40. The wing assembly 100 includes a central section 102 having a bore 104 and two wing members 106 that extend in generally diametrically opposite directions from the central section 102. The central section 102 is a snap fit on the front end 46, being retained by resilient legs 108. Resilient legs 110 on the central section 102 engage in recesses 112 on the main body 40 and prevent rotation of the wing assembly 100 about the longitudinal axis relative to the main body, such as when the wings 106 are held together by the user in a generally vertical position as the cannula is inserted into the patient.

The assembly 10 is supplied in the extended state, shown in FIG. 1. In this state the cannula 18 extends out of the opening 48. A needle sheath or cap 76 is press fitted over the free end 46 of the housing 12 and shields the cannula 18 until ready for use. The method and location of attachment of the cap 76 over the cannula is not critical. The invention may be used with various caps and therefore need not be a push fit and pull release. If desired the cap 76 may be mounted to the shaft 16, rather than the housing 12. The cap may be bonded to the housing 12 or shaft 16 rather than being a simple press fit.

In the extended state the spring 52 is compressed and so biases the needle sub assembly 14 toward the rear end. However, this is prevented by the legs 30, and in particular surfaces 34, engaging the surfaces 35 on the end of the end body 42, as seen in FIG. 8. The protrusions 36 extend into the aperture 74 and prevent the legs 30 extending outwards. Protrusions 75 and 77 on the shaft 16 locate the shaft generally centrally in the aperture 74 and limit off axis movement. Without these protrusions, and in particular protrusions 75, movement off axis could result in unintended release of the legs and retraction of the needle sub assembly.

As supplied the assembly 10 includes a protective shield 80. This shield 80 has a cover portion 82 and two pairs of legs 84 and 86 that extend from the sides of the cover portion 82. The first pair of legs 84 extends into the slots 72 between the legs 70 and the body of the end body 42. The legs 84 extend past the legs and overlie portion 90 of the end body. The legs 84 prevent the legs 70 being squeezed inwards and squeezing legs 30 on the shaft 16 whilst the shield 80 is in place. Thus accidental release of the needle sub assembly 14 from the extended position is prevented whilst the shield 80 is in place.

The second pair of legs 86 extends downwards behind the end of the end body 42. Each leg 86 has an inward extending protrusion 88 that clips around the tubing 22. The legs 86 and protrusions 88 thus hold the shield 80 in place against accidental removal. Removal thus needs to be an intentional action. When the shield 80 is lifted off the end body 42 it is still clipped to the tubing 22 and so will slide back on the tubing but will not fall to the floor. The shield 80 may be removed from the tubing by pulling it off the tubing or can be disposed of with the needle assembly once the needle assembly has been finished with.

With the shield 80 in place the cannula may be inserted into the skin of a patient and taped in place. This can be done with or without shield 80, due to the need to press both of the legs 70 inwards to release the sub assembly 14. However, leaving the shield in place provides an additional level of safety.

The cannula 18 may be inserted into a patient by holding the housing 12 or using the flexible wings 100. As the cannula is inserted, force is transmitted between the cannula 18 and the housing 12 via shaft 16 and legs 30. The legs 30 are sized that they will not buckle or otherwise deflect to allow movement of the cannula 18 toward the retracted position.

When the cannula 18 needs to be removed the user removes the shield 80 from the end body 42 (if it has not already been removed). This allows the legs 70 to be squeezed inwards by the user, using a pinching or squeezing action. The legs 70 are squeezed inwards and contact the legs 30 on the shaft. Continued movement inwards deflects these legs 30 inwards until the surfaces 34 on the end of the legs no longer overlie the opposed surfaces 35 on the end of the end body 42. At that instance the legs 30 are free to move through the opening in the end body 42 and the spring 52 moves the shaft 16 backwards, retracting the cannula 18 from the patient and into the housing 14. The assembly may then be lifted from the patient's skin and disposed of.

It is necessary that both of the legs 70 be squeezed inwards together so as to deflect both of the legs 30 inwards. If only one leg 30 is squeezed inwards the surface 34 on the end of the other leg 30 will still overlie the opposed surface 35 on the end of the end body 42 and so prevent the spring 52 retracting the shaft 16.

Because the needle sub assembly is held in the extended position by the legs 30 during insertion into the patient, the strength of the spring 52 has no relevance during the insertion phase. Thus the spring may be made sufficiently strong to withdraw the cannula 18 from the patient without help from the user.

Thus the user does not need to actively pull the needle sub assembly 14 from the patient's skin, with the cannula point 26 exposed, with the resultant risk of needle stick injury. Further the cannula point 26 is never exposed to the user and there is no cap, sheath or the like to be applied to cover the cannula. Accordingly, other forms of needle stick injury are not possible.

The retraction of the cannula 18 is a linear motion and does not require the user to actively pull on part of the assembly 10 or to hold the housing whilst pulling the needle sub assembly. Thus the risk of the cannula 18 being twisted or withdrawn at a different angle compared to than the angle it was inserted as it is removed is greatly reduced, if not eliminated totally.

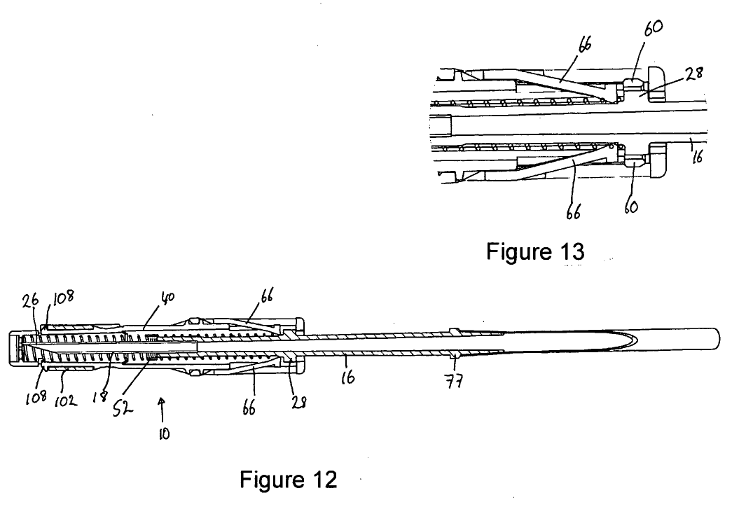

The spring 52 pushes the shaft 16 rearwards until the flange 28 contacts the ends 68 of the legs 66 of the end body 42. As the shaft moves backwards the flange 28 pushes the legs 66 outwards, continues its backwards motion and passes the legs 66, which then snap back towards their unreflected state against the shaft 16. Movement continues until the flange 28 contacts the end of the end body 42. In this position the cannula point 26 is located wholly within the housing 14, as seen in FIGS. 12 & 14 and the flange is located between the end of the end body 42 and the ends of the legs 66.

The legs 66 may engage the spring, but because the flange 28 extends radially more than the spring there will be an outer portion of the flange 28 that extends radially beyond the position of the legs 66. Attempting to extend the cannula 18 will result in the flange 28 contacting the ends 68 of the legs 66. The legs 66 are sized so that normal “accidental” force will resist and prevent any significant movement. If a user deliberately attempts to extend the cannula by application of excessive force the angle of the legs will cause the legs to deflect inwards rather than deflect outwards.

Thus once the cannula has been retracted it cannot be accidentally or easily extended again and either reused or merely exposed.

In the preferred embodiments there is a small gap between the end of the legs and the inner end of the end body and this space is sized to accommodate the flange 28. Thus once the cannula has been fully retracted the shaft is locked in position with relatively little free play.

In the preferred embodiments, in the undeflected state, the free ends of the legs extend to be radially inwards more than the cross bars 60 that define the end of the slots 58. If a user attempts to remove the end body from the main body, as the end body is moved backwards, the free ends of the legs 68 are drawn backwards and contact the cross bars. This prevents separation of the end body 42 from the main body 40. If excessive force is applied the angle of the legs will tend to cause the cross bars 60 to ride over the legs 66 trapping them in the closed slot 58 and preventing separation.

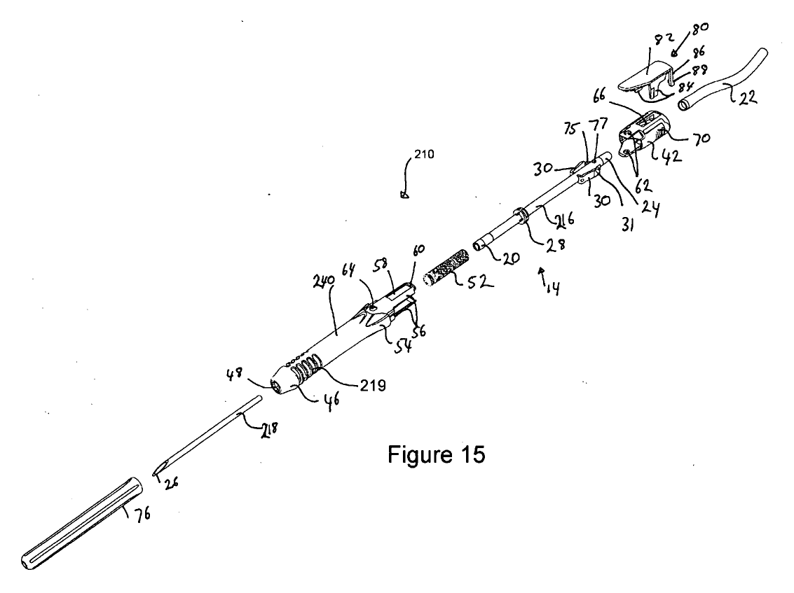

A second example of the invention, comprising a whole blood needle 210, is shown in FIG. 15. The whole blood needle 210 is substantially the same as the first example 10 expect that the main body 240 is substantially longer that the first example and, accordingly, the shaft 216 and cannula 218 are longer.

The whole blood needle 210 does not have a wing sub assembly toward the free end and accordingly lacks the retention legs 118 and recesses 112 of the first example. Instead finger grips 219 are provided toward the free end 46. The other components and features of the whole blood needle 210 are substantially the same as the first example and so the same numbers are used for the same features. The operation of the second example is the same as the first example and the description of the first example is applicable to the second example.

Whilst a single flange 28 is used both as an abutment for the spring 52 and to engage the legs 68 on the end body 42, it will be appreciated that two separate flanges or other structures may be provided.

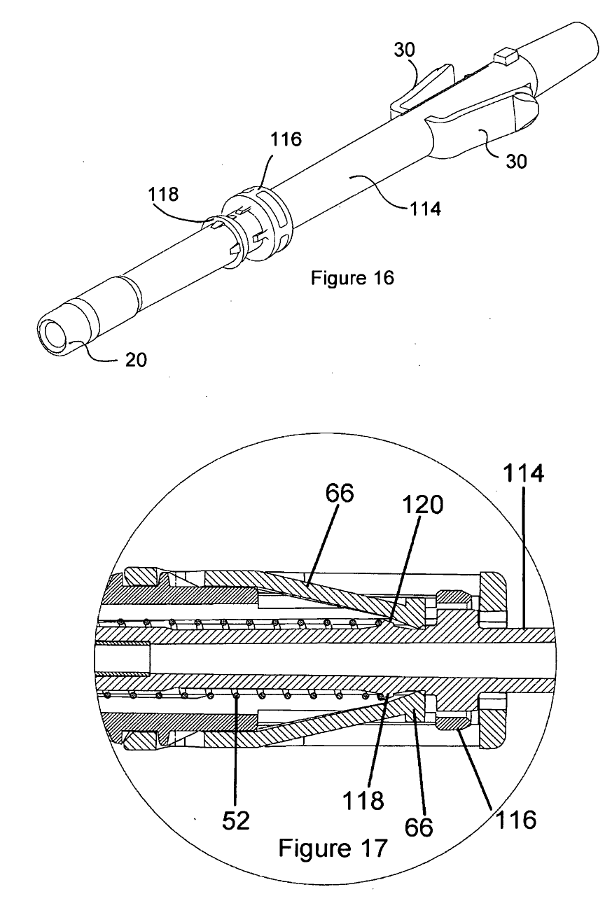

FIGS. 16 and 17 show a shaft 114 that is a variation of the shaft 16 of the first example. Similar parts are numbered the same. The shaft 114 includes a first flange 116 and a second flange 118, located between the first flange 116 and the front end 20. The first flange 116 is substantially the same diameter and provides the same locking function as the flange 28 of the first example, as discussed later. The second flange 118 is of a smaller diameter and acts as a retainer for the spring 52. As seen in FIG. 17, the spring 52 bears against the flange 118 and not against the first flange 116.

FIG. 17 shows the rear portion of the needle assembly when in the retracted state. As seen, the first flange 116 ties between the rear end of the housing and the legs 66. Movement to extend the cannula out of the housing is prevented as previously described.

The second flange 118 is spaced along the shaft 14 a sufficient distance that in this fully retracted the legs 66 lie between the two flanges 116 & 118. Thus the flange 118 and spring 52 are distant from the engagement of the ends of the legs 66 with the flange 116 and so cannot affect the engagement of these components together.

The rear of the flange 118 is provided with a frusto conical circumferential surface 120 that allows the flange 118 to lie closer to the legs 66 in the retracted position than otherwise. Whilst preferred, this is not essential. If desired the circumferential surface 120 may extend parallel to the axis of the shaft 114. This would require the flange 118 to be spaced further from the flange 116 to clear the legs 66.

It will be appreciated that the legs 30 and 66 may be reversed and extend from the housing and shaft respectively. In each case the legs would need to extend in a forwards direction rather than a rearwards direction, as in the example.

Whilst the preferred forms of the examples utilize resilient legs 30, 66 and release arms 70, it will be appreciated that the invention includes other holding, locking and release mechanisms. For example, the legs 30, 66 and arms 70 may be moved in total between their relevant states rather than just being bent. This movement may be by a sliding or pivoting action, for example. For example, the release arms 70 may be separate components slideably mounted on the housing and slide to push or pivot the legs 30 to the release position. Similarly, the legs 30 may be pivotably mounted on the shaft and pivoted by the release mechanism. Alternatively they may slide into a recess to reduce their effective size.

Whilst a pair of opposed legs 30, and associated legs 70 are provided, the invention includes the use of more than two legs 30. Further the legs need not be opposed. As an example, the legs 30 could be located at 90 degrees to each other. Release would thus require two substantially simultaneous pinching actions, such as by use of both hands, further reducing the likelihood of accidental auto-retraction. It will be appreciated that the legs 66 would need to be located in the two unoccupied quadrants around the shaft 16.

Whilst the preferred examples of the invention utilize the shield and legs 30, 70, it will be appreciated that it is not essential that both sets of features be used together. By using the shield to protect the release mechanism, it is possible, although not necessarily desirable, to utilize a single leg 30 to hold the needle sub assembly in the extended state. The presence of the shield prevents accidental auto-retraction.

Similarly, the use of two sets of legs 30, 70 substantially prevents accidental auto-retraction even when the shield is not present, and if desired, embodiments may be supplied without the shield.

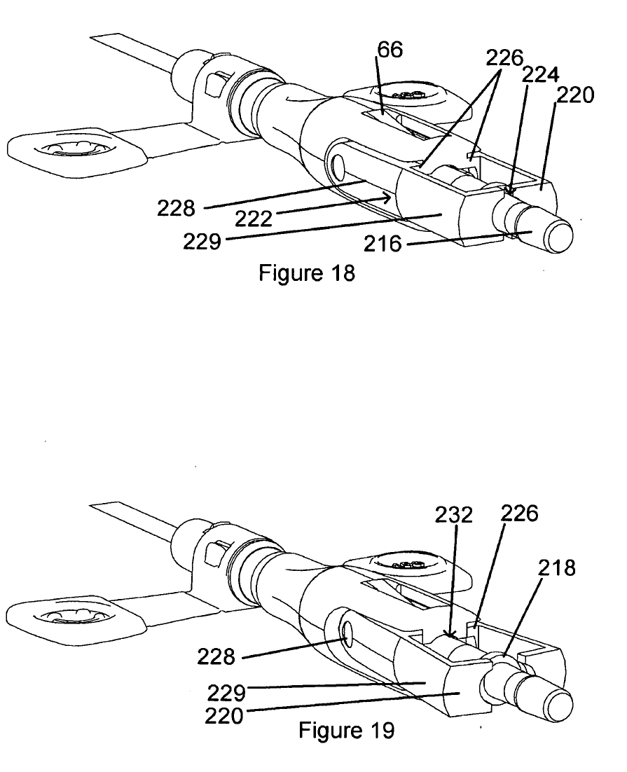

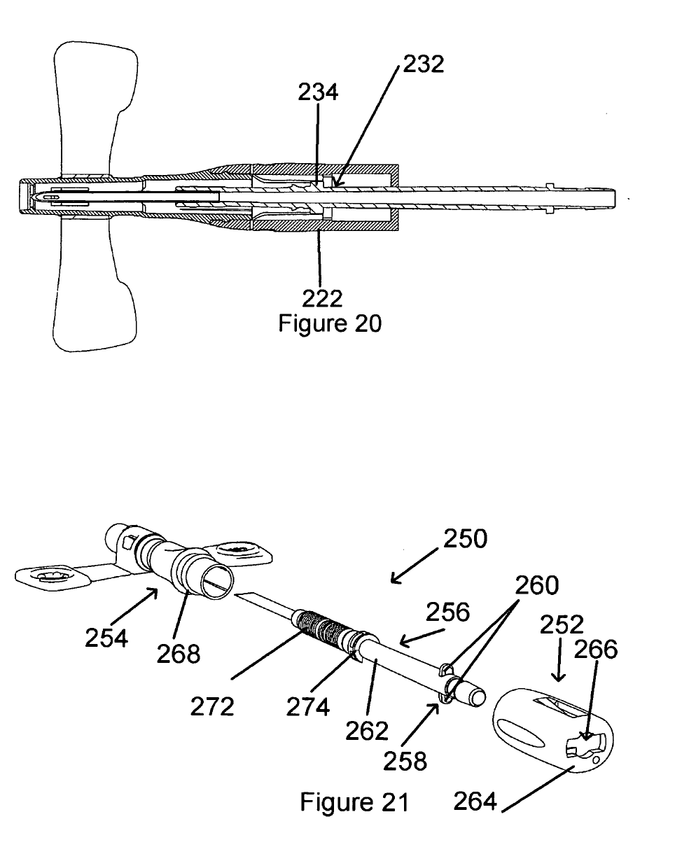

The needle sub assembly may be released by increasing the effective size of the aperture through which the shaft extends. FIGS. 18 to 20 show a further example in which the shaft 216 has a fixed annular flange 218. The end 220 of the housing is defined in part by two opposed movable members 222 that define an aperture 224 between their ends 220 through which the end of the shaft 216 passes. The flange 218 bears against the ends 220. The members 222 are pivotably mounted to the rest of the housing or end cap at hinges 226 so that inner and outer portions 228, 229 extend generally axially on either side of the hinges. The members 226 may be integral with the rest of the housing or end cap with the hinges being living hinges.

To retract the needle sub assembly the user simultaneously presses the inner portions 228 inwards. As shown in FIG. 19, the two members 222 pivot about the hinges 226 and the aperture 224 is enlarged, releasing the flange 218 and allowing the needle sub assembly to be retracted as previously described.

As shown in FIGS. 18 to 20, there may be provided a second aperture 232 located forwards of the end of the housing that retains the shaft. Once the needle sub assembly retracts, rearwards motion is limited by a central flange 234 bearing against the surrounding of this second aperture 232. Forward motion will be prevented by legs 66 as previously described.

Alternatively, by making the aperture 224 with a width smaller than the width of a central flange on the shaft, that central flange will be retained in the housing. Alternatively by ensuring the maximum size of the aperture 224 is less than the size of the central flange, the central flange will be retained.

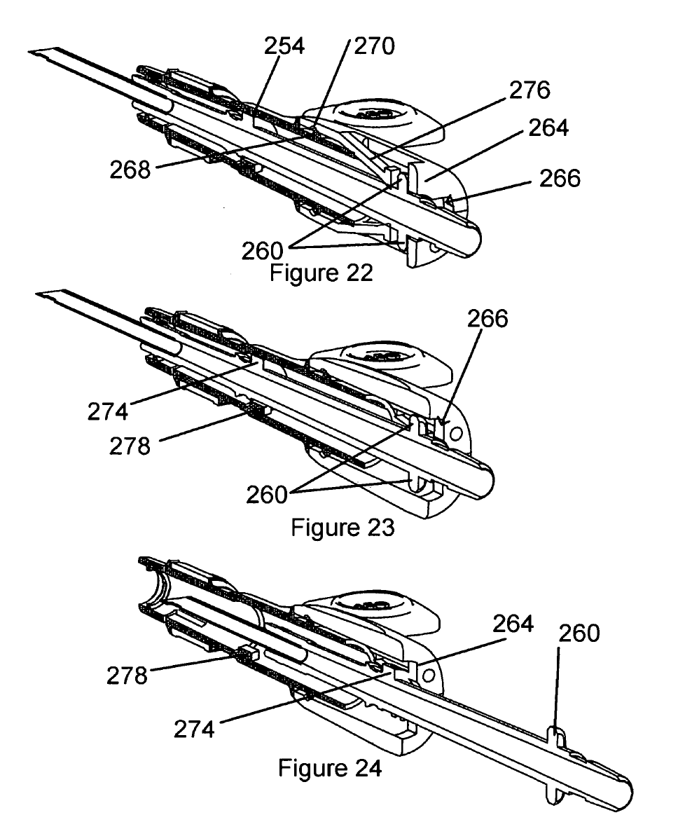

FIGS. 21 to 24 show a retractable needle 250 according to further example in which the housing includes an end cap 252 rotatably mounted to a main body 254. The needle subassembly 256 is slideably mounted within the housing and locking in the retracted position is substantially as previously described.

The needle sub assembly 256 has a non circular retainer 258 at its rear end. In this example the retainer comprises two arms 260 that extend in diametrically opposite direction from the hollow shaft 262.

The end wall 264 of the end cap 252 has an aperture 266 that corresponds to the shape of the retainer 260.

The end cap is a snap fit on the main body and is retained by complimentary circumferential ridge 268 on the main body and circumferential groove 270 on the end cap.

In the extended state the end cap is positioned so the aperture 264 does not align with the retaining 260 and the arms 260 of retainer 258 bear against the end wall 264 of end cap 252.

To retract the cannula, the user rotates the end cap 252 to align the aperture 266 with the retainer 260. At this point the retainer 260 is free to move through the aperture 266 under the action of the spring 272, as described with reference to other examples.

As with the other examples the hollow shaft has a flange 274 that cannot pass through the aperture 264. The spring 272 drives the shaft backwards until the flange 274 contacts the end wall 264.

The end cap 252 has rearwards projecting locking legs 276 that operate in a similar manner to the locking legs of other examples to engage the front surface of flange 274 to prevent forward movement of the needle sub assembly.

To prevent accidental rotation of the end cap detent members may be formed on the end cap and main body to resists rotation. These detent members may be sized so that they need to be broken, with a reasonable force, so that rotation and retraction needs to be conscious action by the user. It will also be appreciated that the needle sub assembly will have a shape such that rotation within the housing is prevented.

Unless the context clearly requires otherwise, throughout the description the words ‘comprise’, ‘comprising’, and the like are to be construed in an inclusive sense as opposed to an exclusive or exhaustive sense; that is to say, in the sense of “including, but not limited to”.

It will be apparent to those skilled in the art that many obvious modifications and variations may be made to the embodiments described herein without departing from the spirit or scope of the invention.

1. A needle assembly including:
an elongate housing having a first aperture in a front end portion thereof and a second aperture in a rear end portion thereof;
an elongate hollow needle sub assembly slideably mounted within the elongate housing and having a needle point at a first end portion thereof and a second end portion adapted to be connected to a supply tube;
the needle sub assembly movable relative to the housing between an extended position, wherein the first end portion extends out of the front of the housing through the first aperture and the needle point is located outside of the housing and a retracted position wherein the first end portion and the needle point is located and shielded within the housing;
biasing means biasing the needle sub assembly to the retracted position;
holding means having an operative position that, when the needle sub assembly is in the extended position, holds the needle sub assembly in the extended position against the action of the biasing means and prevents movement the needle sub assembly toward the retracted position;
release means movable to a release position for releasing the holding means from the operative position to allow movement from the extended to the retracted position;
locking means for locking the needle sub assembly when in the retracted position and preventing movement of the needle sub assembly toward the extended position to expose the needle point, and
means for preventing accidental activation of one or both of the release means or the holding means.

2. The needle assembly of claim 1 wherein the means for preventing accidental activation of the holding means includes the holding means having at least two movable holding members, movable from an operative position to a retracted position, wherein the holding means is operative when at least one of the movable holding members is in the operative position.

3. The needle assembly of claim 1 wherein the means for preventing accidental activation of the holding means includes the release means having at least two movable release members, movable to a release position, wherein the holding means is released from the operative position when two or more release members are in the release position.

4. The needle assembly of claim 1 wherein the means for preventing accidental activation of the holding means includes blocking means that prevents movement of the release means toward the release position.

5. The needle assembly of claim 1 wherein the holding means includes:
at least one protrusion that extends from one of the needle sub assembly and the housing toward the other and
second engagement means on the other of the needle sub assembly and the housing engaged by the at least one protrusion when the holding means is in the operative position.

6. The needle assembly of claim 5 wherein the holding means is released from the operative position by movement of the at least one protrusion.

7. The needle assembly of claim 5 wherein the at least one protrusion comprises at least one second leg located on the needle sub assembly and extending rearwards and outwards toward the housing and having a free end portion that engages said second engagement means on the housing.

8. The needle assembly of claim 5 wherein the second engagement means comprises a portion of the housing surrounding the second aperture.

9. The needle assembly of claim 8 wherein movement of the release means to the release position causes the free end portion of the at least one second leg to move radially inwards to a release position and the second aperture is sized to allow the at least one second leg to pass through when the free end portion is in the release position.

10. The needle assembly of claim 5 wherein the at least one second leg is resilient.

11. The needle assembly of claim 5 wherein the holding means is released from the operative position by movement of the engagement means.

12. The needle assembly of claim 11 wherein movement of the engagement means increases the size of the second aperture to allow the at least one protrusion to pass therethrough.

13. The needle assembly of claim 1 wherein the holding means includes at least a pair of diametrically opposed protrusions on the needle sub assembly, both of which must pass through the second aperture substantially simultaneously during retraction of the needle sub assembly.

14. The needle assembly of claim 1 wherein the release means includes at least one release member for moving at least part of the holding means.

15. The needle assembly of claim 1 including a plurality of second legs and a like number of release members each of which overlays a respective second leg when the needle sub assembly is in the extended state.

16. The needle assembly of claim 15 wherein the release members comprise part of the housing, at least a portion of each release member being movable to move a respective second leg to a release position.

17. The needle assembly of claim 15 wherein the release members comprise part of the housing and movement of each release member bends a respective second leg to a release position.

18. The needle assembly of claim 1 wherein the at least one release member moves a portion of the housing to release the needle subassembly.

19. The needle assembly of claim 1 wherein the locking means includes at least one first leg that extends from one of the needle sub assembly and the housing toward the other and first engagement means on the other of the needle sub assembly and the housing.

20. The needle assembly of claim 1 wherein at least one first leg extends in the longitudinal direction.

21. The needle assembly of claim 1 wherein the first engagement means comprises at least one flange.

22. The needle assembly of claim 1 wherein movement of the needle subassembly from the extended to retracted position causes the at least one leg to pass the engagement means, the at least one first leg being moved to allow said movement and the returning to its original state to prevent movement from the retracted position to the extended position.

23. The needle assembly of claims 22 wherein the at least one first leg is resilient and the at least one first leg is bent to allow movement of the engagement means past the at least one first leg.

24. The needle assembly of claim 1 wherein the at least one first leg comprises at least two opposed first legs located on the housing and extending rearwards and radially inwards toward the needle sub assembly.

25. The needle assembly of claim 1 wherein the engagement means comprises at least one flange on the needle sub assembly and the second aperture is sized so the at least one flange cannot pass therethrough.

26. The needle assembly of claim 1 wherein the assembly includes:
at least one volume into which the release means moves to release the holding means, and
wherein the blocking means extends into at least part of the volume and blocks movement of the release means into the at least one volume.

27. The needle assembly of claim 1 wherein the blocking means comprises a shield that has a cover portion and two blocking arms, the blocking arms configured to extend into a volume with a part of each release arm located on one side thereof and a part of the housing and the holding means on the other side thereof.

28. The needle assembly of claim 1 wherein:
the housing comprises a hollow tubular body having said second aperture at the rear end thereof through which the rear end portion of the needle sub assembly extends;
the holding means comprises two diametrically opposed first legs that extend radially outwards and towards the second end from the shaft and engage the rear end of the housing surrounding the second aperture;
the housing has two diametrically opposed arms that overlie the first legs when the needle sub assembly is extended and which may be squeezed together to compress the legs inwards and allow the legs to pass through the second aperture;
the housing has two opposed locking legs that extend rearwards and inwards and, in the locking position, are located in front of the front surface of a flange on the needle sub assembly to retain the needle sub assembly in the retracted position.
